(19) United States
(12) Patent Application Publication
(10) Pub. No.: US 2009/0105797 A1
(43) Pub. Date: Apr. 23, 2009

(54) EXPANDABLE STENT

(75) Inventors: Blayne A. ROEDER, Lafayette, IN (US); Alan R. Leewood, Lafayette, IN (US); David D. Grewe, West Lafayette, IN (US)

Correspondence Address: BRINKS HOFER GILSON & LIONE/CHICAGO/COOK, PO BOX 10395, CHICAGO, IL, 60610, US

(73) Assignee: MED Institute, Inc., West Lafayette, IN (US)

(21) Appl. No.: 12/250,957

(22) Filed: Oct. 14, 2008

Related U.S. Application Data
(60) Provisional application No. 60/980,999, filed on Oct. 18, 2007.

(51) Int. Cl. A61F 2/86

(52) U.S. Cl. 623/1.2

(57) ABSTRACT

A stent includes a radially expandable tubular structure having a first end, a second end, and a primary strut arrangement extending over substantially an entire length thereof. The primary strut arrangement includes a plurality of rows of struts. The struts are interconnected within each row in a sinusoidal arrangement about a circumference of the tubular structure. Crests and troughs in the sinusoidal arrangement include connection points of the struts. A plurality of longitudinal struts connect neighboring rows of struts at the connection points. In each row, four circumferentially adjacent struts are disposed between every two longitudinal struts joined to the row. One of the two longitudinal struts extends in a direction of the first end to a first neighboring row, and the other of the two longitudinal struts extends in a direction of the second end to a second neighboring row.

### RELATED APPLICATIONS

The present patent document claims the benefit of the filing date under 35 U.S.C. §119(e) of U.S. Provisional Patent Application Ser. No. 60/980,999, which was filed on Oct. 18, 2007, and is hereby incorporated by reference in its entirety.

### TECHNICAL FIELD

The present disclosure relates generally to medical devices and more particularly to expandable stents.

### BACKGROUND

Stents are generally designed as tubular support structures that can be used in a variety of medical procedures to treat blockages, occlusions, narrowing ailments and other problems that restrict flow through body vessels. Expandable stents are radially compressed for delivery within a vessel and then radially expanded once in place at a treatment site, where the tubular support structure of the stent contacts and supports the inner wall of the vessel. Such stents are generally classified as either balloon-expandable or self-expanding. Balloon-expandable stents expand in response to the inflation of a balloon, while self-expanding stents expand spontaneously when released from a delivery device.

Numerous vessels throughout the vascular system, including peripheral arteries, such as the carotid, brachial, renal, iliac and femoral arteries, and other vessels, may benefit from treatment by a stent. For example, the superficial femoral artery (SFA) may be a site of occlusions or blockages caused by peripheral artery disease. This condition causes leg pain and gangrene in severe cases and affects roughly 8 million to 12 million Americans according to the American Heart Association.

Due to its location in the vicinity of the hip joint, the SFA may experience repetitive axial strains that can cause the artery to elongate or contract up to 10-12%. Stents placed in the SFA may thus be prone to fatigue failure. A major challenge of treating the SFA is providing a stent having sufficient axial flexibility and excellent fatique properties to withstand the recurring axial strains of the arterial environment. The inventors believe that presently available stents do not provide these advantages.

### BRIEF SUMMARY

Described herein is an expandable stent that may provide advantages over presently available stents for use in the SFA and other vessels.

The stent includes a radially expandable tubular structure having a first end, a second end, and a primary strut arrangement extending over substantially an entire length thereof from the first end to the second end. The primary strut arrangement includes a plurality of rows of struts. The struts are interconnected within each row in a sinusoidal arrangement about the circumference. Crests and troughs in the sinusoidal arrangement include connection points of the struts, and the sinusoidal arrangements of the rows are in phase with each other. A plurality of longitudinal struts connect neighboring rows of struts at the connection points. In each row, four circumferentially adjacent struts are disposed between every two longitudinal struts joined to the row. One of the two longitudinal struts extends in a direction of the first end to a first neighboring row, and the other of the two longitudinal struts extends in a direction of the second end to a second neighboring row.

### BRIEF DESCRIPTION OF THE DRAWINGS

FIG. 1A is a perspective view of a radially expanded stent according to one embodiment, where the stent has a primary strut arrangement;

FIG. 1B is a flattened plan view of a portion of the primary strut arrangement of the stent of FIG. 1A;

FIG. 1C is an enlarged view of a portion of the primary strut arrangement shown in FIG. 1B;

FIG. 2 is a flattened plan view of a radially expanded stent according to another embodiment, where the stent has a primary strut arrangement, a secondary strut arrangement, and a tertiary strut arrangement;

FIG. 2A is a close-up view of four circumferentially adjacent struts from the secondary strut arrangement of FIG. 2;

FIG. 2B is a close-up view of four circumferentially adjacent struts from the tertiary strut arrangement of FIG. 2;

FIG. 3 is a flattened plan view of a radially expanded stent having the primary and secondary strut arrangements of FIG. 2 and a tertiary strut arrangement according to another embodiment;

FIG. 3A is a close-up view of four circumferentially adjacent struts from the tertiary strut arrangement of FIG. 3;

FIG. 4 is a flattened plan view of the stent of FIG. 2 in an unexpanded configuration;

FIG. 5A is a flattened plan view of a portion of a presently available stent in a radially expanded configuration;

FIG. 5B is a flattened plan view of the portion of the presently available stent of FIG. 5A subjected to an axial extension of 20%;

FIG. 6A is a flattened plan view of a portion of an improved stent according to one embodiment in a radially expanded configuration;

FIG. 6B is a flattened plan view of the portion of the improved stent of FIG. 6A subjected to an axial extension of 20%;

FIG. 7 shows a T-bar structure of the presently available stent of FIG. 5B; and

FIG. 8 shows a T-bar structure of the improved stent of FIG. 6B.

### DETAILED DESCRIPTION

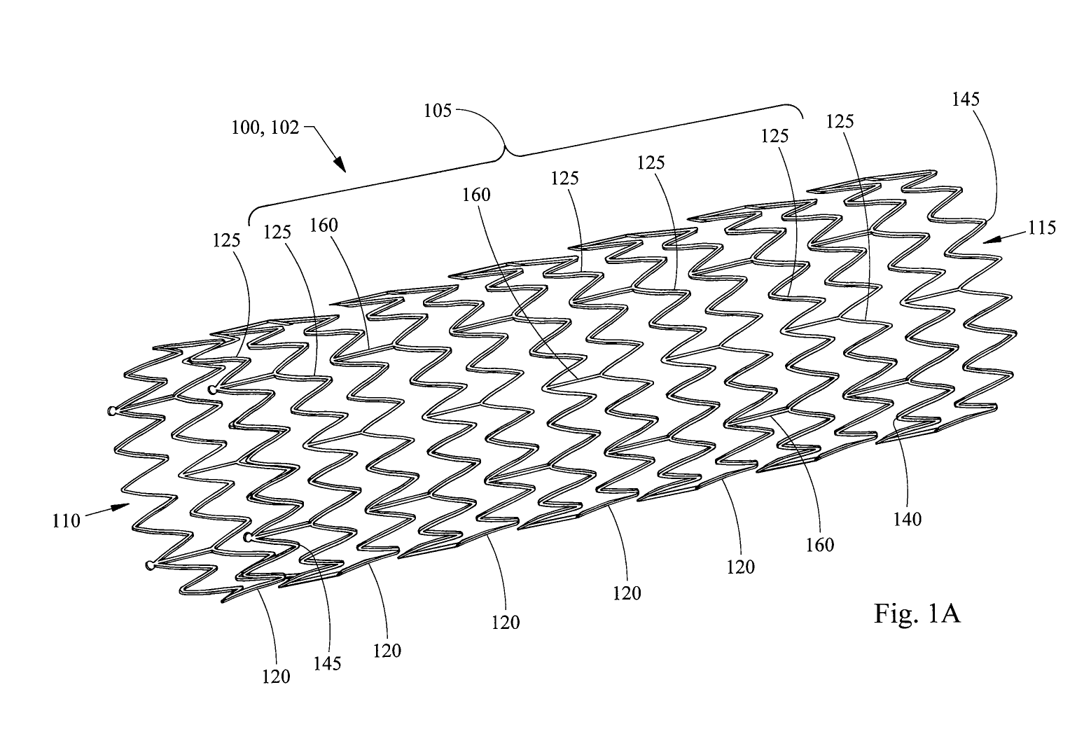

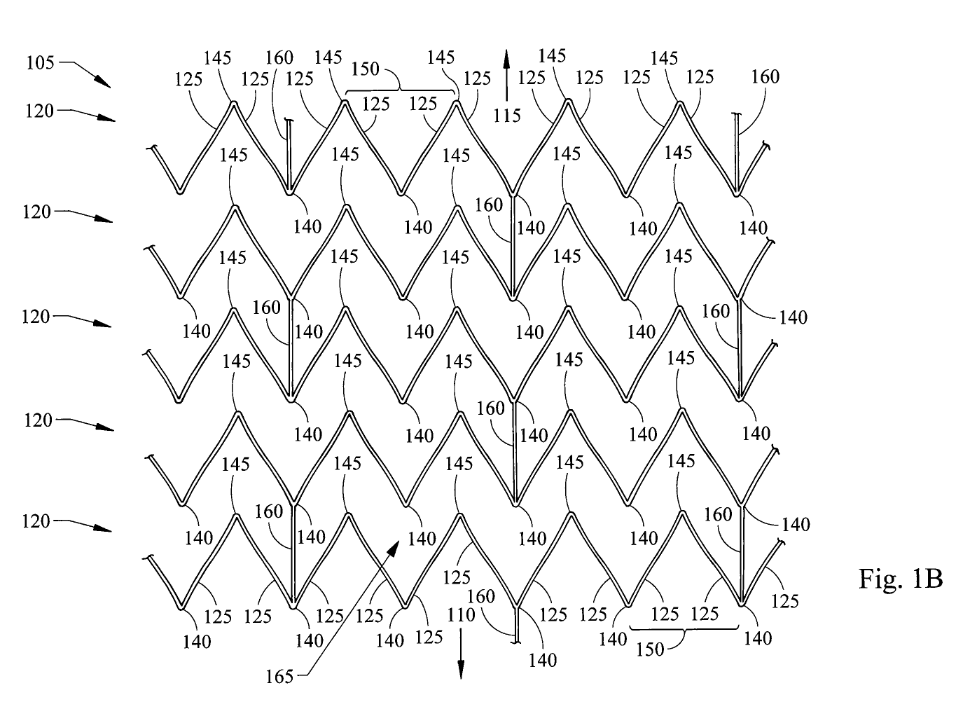

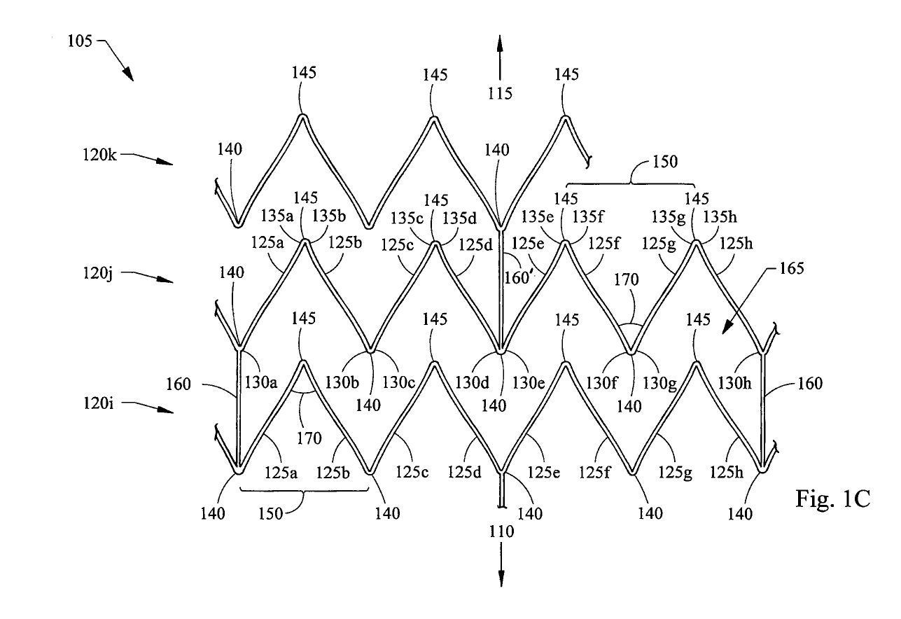

FIG. 1A shows an expandable stent 100 according to a first embodiment. The stent 100 includes a thin-walled tubular structure 102 having a first end 110 and a second end 115. The first end 110 could be either a distal end or a proximal end of the stent 100. Similarly, the second end 115 could be either the distal or proximal end of the stent 100. The distal end of a stent is the end that enters a body vessel and reaches the treatment site first, and the proximal end is the trailing end of the stent. Consequently, a distal direction generally refers to the direction in which the stent is moving within a body vessel or to the direction of the distal end of the stent. A proximal direction is opposite to the distal direction.

Referring to FIGS. 1A and 1B, the stent 100 includes a primary strut arrangement 105 extending over substantially an entire length of the tubular structure 102 from the distal end 110 to the proximal end 115. FIG. 1A is a perspective view of a stent 100 according to a first embodiment in a radially expanded configuration, and FIG. 1B is a flattened plan view of a portion of the primary strut arrangement 105 of the stent 100. Preferably, the primary strut arrangement 105 spans at least 80% of the length of the tubular structure 102. The primary strut arrangement 105 may also extend over at least 90% of the length of the tubular structure 102.

Referring to FIG. 1B, the primary strut arrangement 105 includes a plurality of rows 120 of struts 125. Each row 120 is disposed about a circumference of the tubular structure 102. According to one embodiment, the plurality of rows 120 extend from the first end 110 to the second end 115 of the structure 102. The struts 125 are connected to each other within each row 120 in a sinusoidal (e.g., zig-zag) arrangement about the circumference. Troughs 140 and crests 145 of the sinusoidal arrangement include connection points of the struts. Each strut 125 is joined to its two nearest neighbors. Preferably, any two circumferentially adjacent struts 125 have a wishbone shape 150 (i.e., V-shape) when the stent 100 is expanded. The sinusoidal arrangements of the rows are in phase with each other. That is, the troughs 140 of the rows 120 are generally aligned with each other, and the crests 145 of the rows 120 are generally aligned with each other.

The primary strut arrangement 105 further includes longitudinal struts 160 that connect the rows 120 of struts 125. Referring again to FIG. 1B, the longitudinal struts 160 may serve as bridges between troughs 140 in neighboring rows 120. According to one embodiment, the longitudinal struts 160 are oriented in a direction parallel to a longitudinal axis of the stent 100 and extend between longitudinally adjacent troughs 140. According to another embodiment, the longitudinal struts 160 may be slanted or angled with respect to the longitudinal axis of the stent 100. The longitudinal struts 160 may be straight, as shown in FIG. 1B, or curved.

Each strut 125 has a first portion oriented closer to the first end 110 and a second portion oriented closer to the second end 115. First portions of the struts 125 are joined to each other at the troughs 140, and second portions of the struts 125 are joined to each other at the crests 145. For example, referring to the middle row 120j shown in FIG. 1C, the second portion 135a of a first strut 125a is joined to the second portion 135b of a second strut 125b that is disposed circumferentially adjacent to the first strut 125a. The first portion 130b of the second strut 125b is joined to the first portion 130c of a third strut 125c that is disposed circumferentially adjacent to the second strut 125b. The second portion 135c of the third strut 125c is joined to the second portion 135d of the fourth strut 125d, and so on. Preferably, four circumferentially adjacent struts (e.g., struts 125a, 125b, 125c, and 125d) are disposed between every two longitudinal struts (e.g., longitudinal struts 160 and 160′) joined to a given row (e.g., row 120j). One of the two longitudinal struts 160 extends in a direction of the first end 110 from a trough 140 in the row 120j to a trough 140 in a neighboring row 120i, and the other longitudinal strut 160′ extends in a direction of the second end 115 from a trough 140 in the row 120j to a trough 140 in another neighboring row 120k.

A first closed cell 165 of the tubular structure 105 includes sixteen struts 125 from two neighboring rows and two longitudinal struts 160 connecting the two rows and bordering the sixteen struts 125. Referring again to FIG. 1C, the first closed cell 165 is shown having sides defined by the two longitudinal struts 160, a first end portion or bottom defined by eight circumferentially adjacent struts 125a-125h from a bottom row 120i, and a second end portion or top defined by eight circumferentially adjacent struts 125a-125h from a top row 120j. Preferably, three or more circumferentially adjacent first closed cells are disposed about the circumference of the structure 120. Accordingly, each row 120 of the primary strut arrangement 105 may include at least twenty-four circumferentially adjacent struts 125. It is also preferred that a plurality of first closed cells 165 extend over substantially an entire length of the tubular structure 102 from the distal end 110 to the proximal end 115.

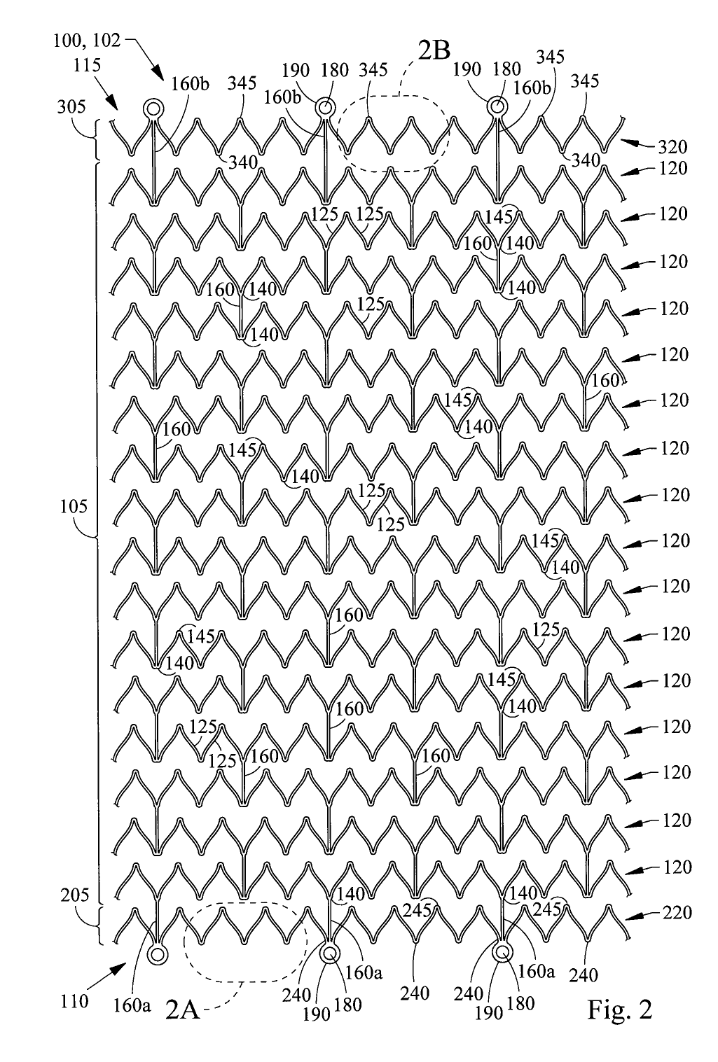

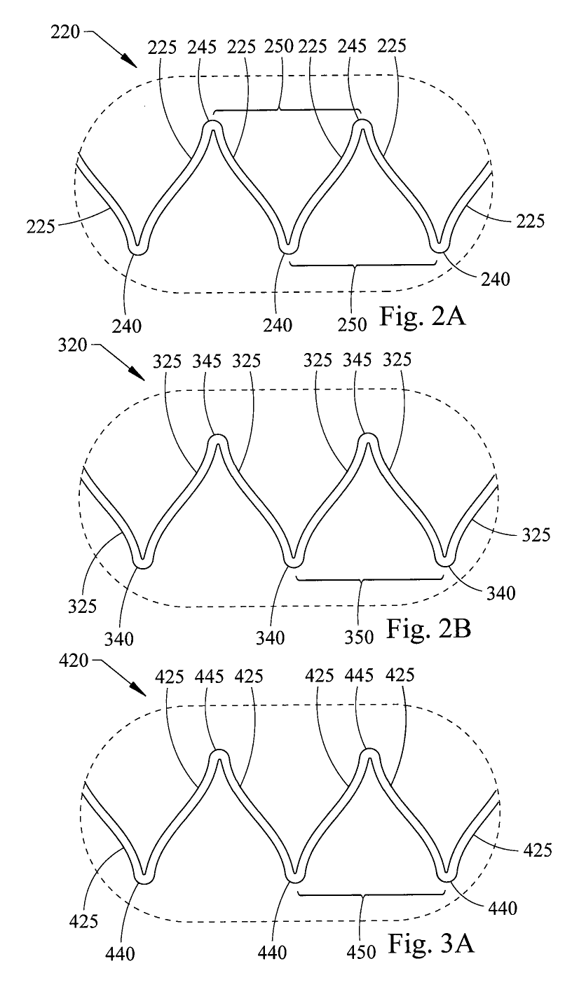

The tubular structure 102 of the stent 100 may include a secondary strut arrangement 205 disposed adjacent to the primary strut arrangement 105, as shown in FIG. 2. The secondary strut arrangement 205 may be disposed at the first end 110 of the structure 102 and may include one or more rows 220 of second struts 225 disposed in a sinusoidal arrangement about a circumference of the structure 102. Referring to FIG. 2A, the second struts 225 are connected to each other at troughs 240 and crests 245 within the row 220, such that each second strut 225 is connected to its two nearest neighbors. Preferably, any two circumferentially adjacent second struts 225 have a wishbone shape 250 when the structure 102 is radially expanded. According to this embodiment, the sinusoidal arrangement of the row 220 of second struts 225 is in phase with the sinusoidal arrangements of the rows 120 of struts 125 of the primary strut arrangement 105.

The longitudinal struts 160a at an end of the primary strut arrangement 105 and extending in a direction of the first end 110 of the tubular structure 102 are joined to the row 220 of second struts 225 at the troughs 240, according to this embodiment. The troughs 240 in the row 220 are disposed closer to the first end 110 of the structure 102 than are the crests 245 in the row 220. Eight circumferentially adjacent second struts 225 may be disposed between every two longitudinal struts 160a joined to the row 220 of second struts 225. The longitudinal struts 160a connect the primary strut arrangement 105 with the secondary strut arrangement 205.

The tubular structure 102 of the stent 100 may further include a tertiary strut arrangement 305 including at least one row 320 of third struts 325 disposed adjacent to the primary strut arrangement 105 at the second end 115 of the tubular structure 102. According to the embodiment of FIG. 2, the tertiary strut arrangement 305 includes one row 320 of third struts 325. The third struts 325 are interconnected within the row 320 in a sinusoidal arrangement about the circumference at crests 345 and troughs 340 in the sinusoidal arrangement.

The longitudinal struts 160b disposed at an end of the primary strut arrangement 105 and extending in a direction of the second end 115 of the tubular structure 102 are joined to the row 320 of third struts 325 at the crests 345, according to this embodiment. The crests 345 in the row 320 are closer to the second end 115 of the tubular structure 102 than are the troughs 340 in the row 320. Preferably, eight circumferentially adjacent third struts 325 are disposed between every two longitudinal struts 160b joined to the row 320. It is also preferred that the longitudinal struts 160b are substantially parallel to a longitudinal axis of the tubular structure 102. The primary strut arrangement 105 is thus connected with the tertiary strut arrangement 305. Preferably, the sinusoidal arrangement of the row 320 of third struts 325 is 180 degrees out of phase with the sinusoidal arrangements of the rows 120 of struts 125 of the primary strut arrangement 105.

The stent may include one or more radiopaque markers attached to one or both ends of the tubular structure 102. For example, as shown in FIG. 2, radiopaque markers 180 may be secured in eyelets 190 integrally formed with the tubular structure 102 at the first end 110 and the second end 115. Preferably, the radiopaque markers 180 are disposed at connection points that include longitudinal struts (e.g., longitudinal struts 160a or 160b).

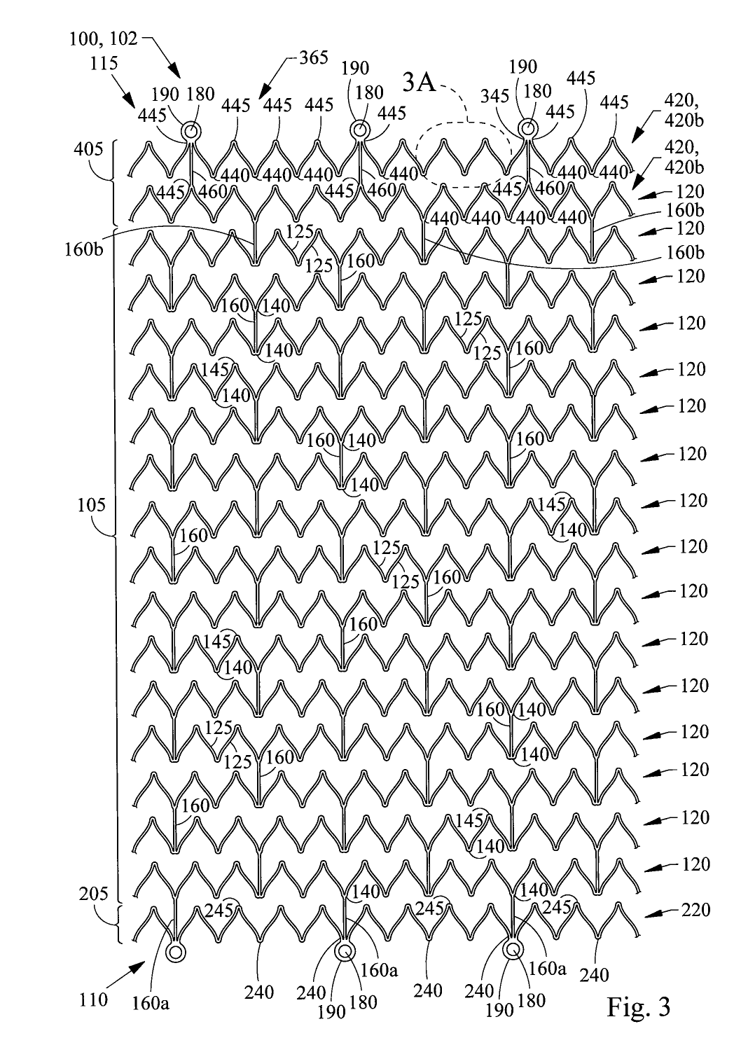

FIG. 3 shows the tubular structure 102 of the stent 100 including a tertiary strut arrangement 405 according to a second embodiment. The primary strut arrangement 105 and the secondary strut arrangement 205 are the same as in the previous embodiment. The tertiary strut arrangement 405 of FIG. 3 includes two rows 420 of third struts 425 interconnected within each row 420 in a sinusoidal arrangement about the circumference. Crests 445 and troughs 440 in the sinusoidal arrangement include connection points of the third struts 425. The crests 445 in each row 420 are closer to the second end 115 of the tubular structure 102 than are the troughs 440 in the row 420, and a second row 420b of the two rows 420 is closer to the second end 115 of the tubular structure 102 than is a first row 420a of the two rows 420. The sinusoidal arrangements of the two rows 420 of third struts 425 are in phase with each other, according to this embodiment. Axial struts 460 connect the two rows 420 of third struts 425 from crests 445 of the first row 420a to crests 445 of the second row 420b. According to this embodiment, in each of the two rows 420, eight circumferentially adjacent third struts 425 are disposed between every two axial struts 460 joined to the row 420. Preferably, the axial struts 460 are substantially parallel to a longitudinal axis of the tubular structure 102.

The longitudinal struts 160b disposed at an end of the primary strut arrangement 105 and extending in a direction of the second end 115 of the tubular structure 102 are joined to the first row 420a of third struts 425 at troughs 440 spaced apart from every axial strut 460 by three third struts 425 or five third struts 425. The primary strut arrangement 105 is thus connected with the tertiary strut arrangement 405. Preferably, the sinusoidal arrangements of the two rows 420 of third struts 425 are in phase with the sinusoidal arrangements of the rows 120 of struts 125 of the primary strut arrangement 105.

The strut arrangements described herein are applicable to stents of any length or diameter. The length of the stent may lie between about 20 mm and about 140 mm. Preferably, the length of stent is about 50 mm or larger. In the expanded configuration, the diameter of the stent may lie between about 4 mm and about 36 mm.

Preferably, the stent is formed of a superelastic (or shape memory) material. Such a material may undergo a reversible phase transformation that allows it to “remember” and return to a previous shape or configuration. For example, superelastic nickel-titanium alloys may transform between an elastically deformable martensitic phase and a stronger austenitic phase by isothermally applying and releasing stress (superelastic effect) and/or by cooling and heating (shape memory effect). In use, the superelastic effect is generally employed for the present stents to substantially recover an original stress-free configuration after significant straining. Austenite has a high yield strength in the range of from 195 MPa to 690 MPa, while martensite may be deformed up to a recoverable strain of about 8%. Preferably, the superelastic or shape memory material is an equiatomic or near-equiatomic nickel-titanium alloy. The nickel-titanium alloy may further include ternary and quaternary alloying elements. For example, the nickel-titanium alloy may include one or more of V, Cr, Mn, Fe, Co, Ni, Cu Zn, Zr, Nb, Mo, Ru, Rh, Pd, Ag, Cd, Hf, Ta, W, Re, Os, Ir, Pt, Au, Bi, and Hg.

Self-expanding stents are typically deployed in the body by utilizing the superelastic effect. The stent may be constrained within a tubular sheath for delivery into a body vessel and then deployed by retracting the sheath, thereby releasing the stress within the stent and allowing the stent to expand. The compressed state or delivery configuration of the stent preferably comprises the martensitic phase of a nickel-titanium alloy, and the expanded state of the stent preferably comprises the austenitic phase. For superelastic deployment of the stent in the body, it is desirable that an austenite finish temperature (A_{f}) of the nickel-titanium alloy is less than or equal to body temperature.

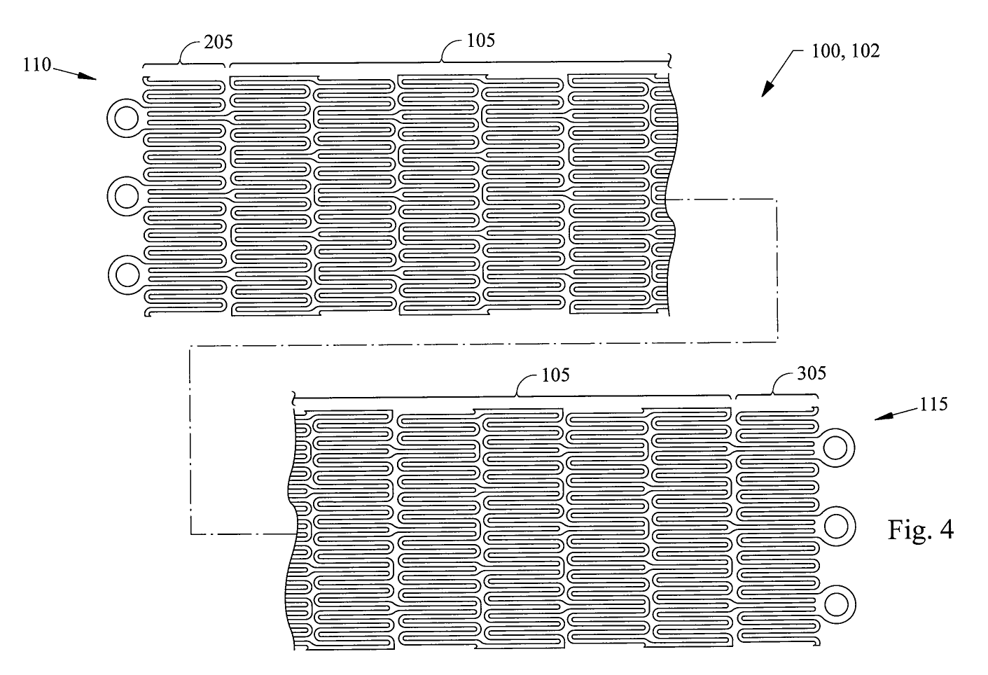

The stent may be laser cut from thin-walled tubes by laser-cutting techniques known in the art. FIG. 4 shows a flattened plan view of a stent 200 laser cut from a tube to have the strut arrangements 105, 205 described in reference to FIG. 2. After laser cutting, the stent may be radially expanded to the desired diameter and heat treated to impart a “shape memory” of the expanded configuration. Typical heat treatment temperatures for nickel-titanium superelastic materials are in the range of from about 400° C. to about 600° C. After heat treatment, the stent displays superelastic or shape memory behavior when exposed to the appropriate stresses and/or temperatures. The heat-treated, expanded stent may be compressed to a delivery configuration similar to that shown in FIG. 4 for insertion into a sheath and delivery into a body vessel. The stent may be cooled to a temperature below a martensite finish temperature (M_{f}) of the superelastic alloy prior to compression to the delivery configuration.

A stent having a primary strut arrangement in accordance with the present disclosure may show excellent axial flexibility and fatigue life, as will be further discussed below.

### EXAMPLES

### 1. Axial Fatigue Tests

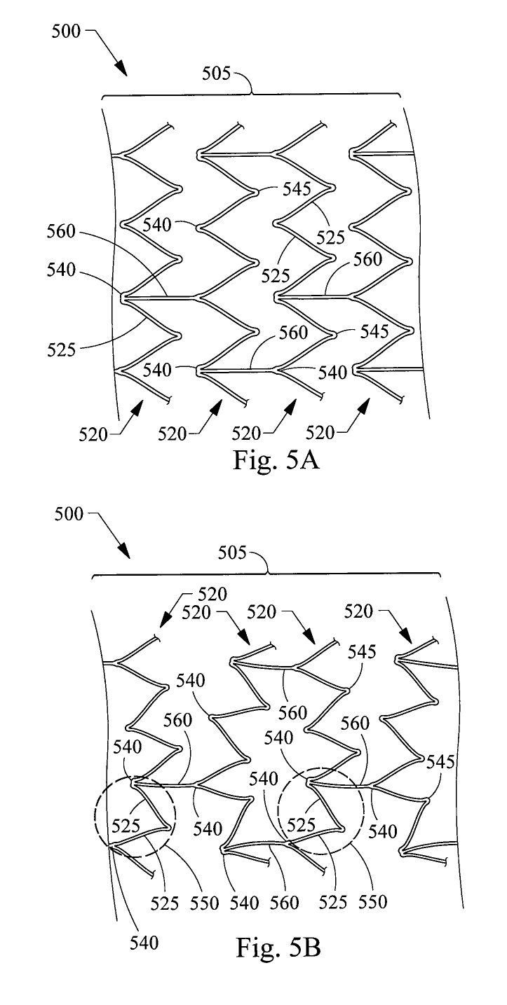

Strain-controlled axial fatigue tests were carried out on self-expanding stents having the primary strut arrangement 105 described herein (“improved stents”). Presently available self-expanding stents having a different primary strut arrangement (“presently available stents”) also underwent fatigue testing under the same strain conditions. The primary strut arrangement 505 of the presently available stents is shown in FIG. 5A. Both the presently available stents and the improved stents had the same nominal dimensions and were made of a superelastic nickel-titanium alloy. Specifically, the stents were designed to fit into a 5 French delivery system and underwent fatigue testing at an expanded diameter of 6 mm and/or 10 mm.

The fatigue testing entailed cyclic application of an axial force to the expanded stents to achieve a given strain half-amplitude in tension and compression, where the strain half-amplitude is equivalent to

$$\frac{Δ L}{L} ,$$

L being equal to the initial length of the stent and ΔL being the change in length upon application of the force. The duration of each fatigue test was 400 million cycles. A series of tests were carried out at different strain half-amplitudes to estimate the maximum strain sustainable by the stent without failure over an infinite number of cycles (i.e., >10⁸ cycles).

The improved stents were able to sustain a strain half-amplitude in tension and compression of ±12% over the duration of the tests without failing. In other words, they exhibited an endurance limit of about 12%. In contrast, the presently available stents exhibited an endurance limit of about 2%; that is, they failed during cycling when the strain half-amplitude exceeded about ±2%.

### 2. Finite Element Analysis

The finite element analysis (FEA) software ABAQUS developed by Dassault Systèmes was employed to analyze the deformation of the improved stents and presently available stents under a hypothetical axial load. The geometry of each stent was modeled using plane stress elements and the material of the stents was assumed to be elastic. Because of the symmetry of the structure, it was possible to to carry out FEA simulations on only a portion of the stents by using appropriate boundary conditions.

During the FEA simulations, each stent was axially extended in tension by 20%, which is over twice the axial deformation the stent is expected to experience in-vivo under the most harsh anatomical conditions. The extreme strain amplitude was selected to accentuate the stent response and to highlight differences between the improved and presently available stents of this size. While FEA simulations can be used to make absolute predictions, the technique is considerably more precise when making relative comparisons as is being done here.

FIG. 5A is a flattened plan view of a portion of a presently available stent 500 having a strut arrangement 505 that was modeled using the ABAQUS software. Circumferentially adjacent struts 525 are joined at troughs 540 and crests 545 within each row 520, and longitudinal struts 560 join neighboring rows 520 of struts 525 at the troughs 540. FIG. 5B shows the FEA simulation results after the stent 500 was subjected to an axial force sufficient to elongate the stent 500 by 20%. The distortion of the strut arrangement due to the axial load is apparent. Regions including a single wishbone structure 550 formed by two circumferentially adjacent struts 525 are highlighted. It is believed that these single wishbone structures 550 transmit localized strains into the longitudinal struts 560, as will be discussed further below.

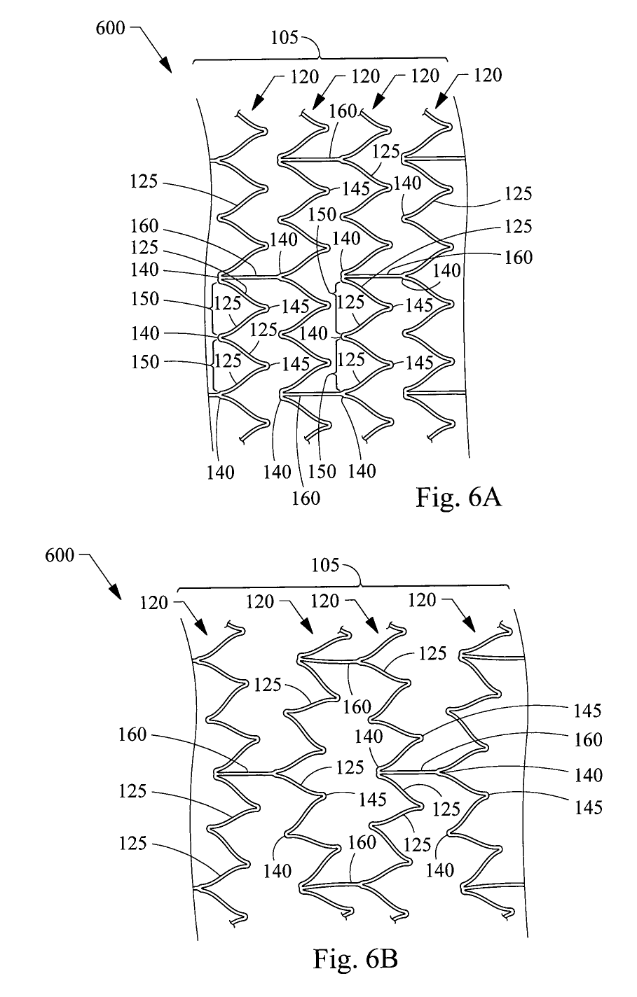

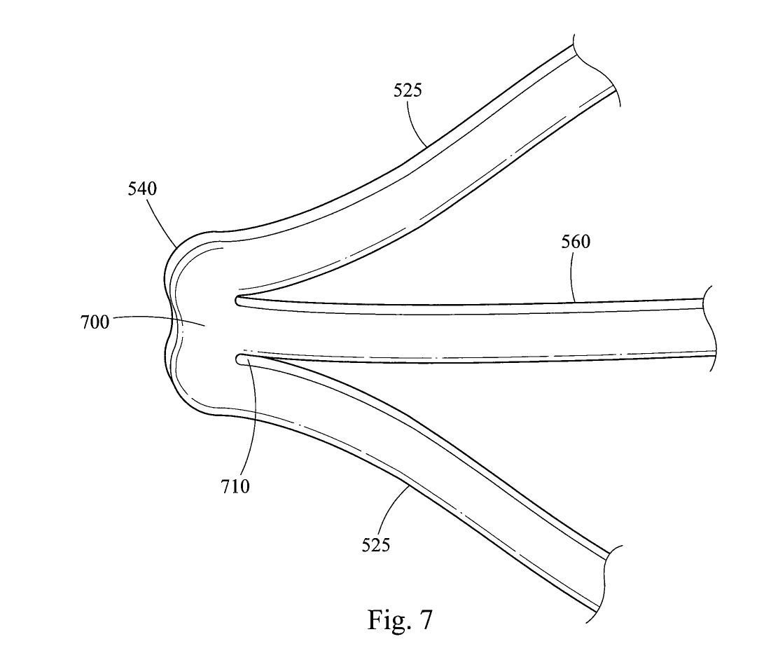

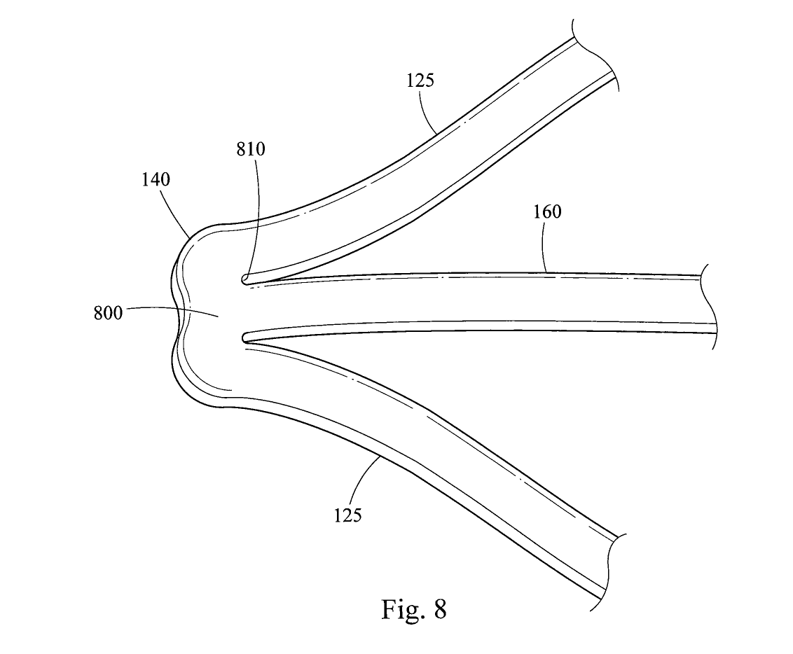

FIG. 6A is a flattened plan view of a portion of the improved stent 600 that was modeled using the ABAQUS software. FIG. 6B shows the FEA simulation results after the stent 600 was subjected to an axial force sufficient to elongate the stent 600 by 20%. As in the previous example, the strut arrangement is distorted due to the axial load. In this case, however, four circumferentially adjacent struts 125, or two wishbone structures 150, lie between every two longitudinal struts 160 joined to a given row 120. It is believed that these two wishbone structures 150 are better able to accommodate the axial strain than the single wishbone structure 550 of the presently available stents 500, and thus the strain transmitted to the junctions of the longitudinal struts 160 and the troughs 140 is reduced. Referring to FIG. 7 or FIG. 8, these junctions may be referred to as T-bar regions 700, 800 due to their geometry.

Strain contour plots showing the spatial distribution of strain in the improved and presently available stents during loading were obtained using the ABAQUS software. Data from the strain contour plots indicate that the strain concentration reaches a maximum at T-bar regions for both the improved and presently available stents. If the strain concentration becomes excessively high in these regions, the stents may be prone to fatigue failures at these sites.

FIG. 7 shows the region 710 of maximum strain during loading in the presently available stent 500, and FIG. 8 shows the region 810 of maximum strain during loading in the improved stent 600. The maximum strains occur in the vicinity of the T-bar regions 700, 800. A maximum strain of about 2.8% occurs in the presently available stent 500, whereas the maximum strain is advantageously reduced by over 60% to about 1.1% in the improved stent 600.

The inventors believe the FEA simulations demonstrate a significant improvement in the stent described herein relative to a previous design due to the substantial decrease in maximum strain, which is known due drive fatigue failures in many materials, including superelastic Nitinol. The results of the FEA simulations and the axial fatigue experiments suggest that the improved stents may have a higher fatigue life than presently available stents. Accordingly, the improved stents may be more appropriate for treating the superficial femoral artery.

Although the present invention has been described in considerable detail with reference to certain embodiments thereof, other embodiments are possible without departing from the present invention. The spirit and scope of the appended claims should not be limited, therefore, to the description of the preferred embodiments contained herein. All embodiments that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A stent comprising:
a radially expandable tubular structure comprising a first end, a second end, and a primary strut arrangement extending over substantially an entire length thereof from the first end to the second end, the primary strut arrangement comprising:
a plurality of rows of first struts, the first struts being interconnected within each row in a sinusoidal arrangement about a circumference of the tubular structure, wherein crests and troughs in the sinusoidal arrangement comprise connection points of the first struts, the sinusoidal arrangements of the rows being in phase with each other; and
a plurality of longitudinal struts connecting neighboring rows of first struts at the connection points, wherein in each row four circumferentially adjacent first struts are disposed between every two longitudinal struts joined to the row, one of the two longitudinal struts extending in a direction of the first end to a first neighboring row and the other of the two longitudinal struts extending in a direction of the second end to a second neighboring row.

2. The stent of claim 1, wherein the primary strut arrangement extends over at least 80% of the length of the structure from the first end to the second end.

3. The stent of claim 1, wherein the primary strut arrangement extends over at least 90% of the length of the structure from the first end to the second end.

4. The stent of claim 1, wherein the length of the stent is at least about 50 mm.

5. The stent of claim 1, wherein the longitudinal struts are substantially parallel to a longitudinal axis of the structure.

6. The stent of claim 1, wherein any two circumferentially adjacent first struts have a wishbone shape when the stent is expanded.

7. The stent of claim 1, wherein the tubular structure includes two or more radiopaque markers secured to the first end and to the second end thereof.

8. The stent of claim 1, wherein the tubular structure is laser cut from a tube.

9. The stent of claim 1, wherein the tubular structure is made of a superelastic material.

10. The stent of claim 1, further comprising a secondary strut arrangement including at least one row of second struts disposed adjacent to the primary strut arrangement at the first end of the tubular structure.

11. The stent of claim 10, wherein the secondary strut arrangement comprises one row of second struts, the second struts being interconnected within the row in a sinusoidal arrangement about the circumference of the tubular structure, wherein crests and troughs in the sinusoidal arrangement comprise connection points of the second struts, and wherein the sinusoidal arrangement of the row of second struts is in phase with the sinusoidal arrangements of the rows of first struts of the primary strut arrangement,
wherein the longitudinal struts at an end of the primary strut arrangement and extending in a direction of the first end of the tubular structure are joined to the row of second struts at the troughs, the troughs in the row being closer to the first end than the crests in the row, eight circumferentially adjacent second struts being disposed between every two longitudinal struts joined to the row of second struts, thereby connecting the primary strut arrangement with the secondary strut arrangement.

12. The stent of claim 10, further comprising a tertiary strut arrangement including at least one row of third struts disposed adjacent to the primary strut arrangement at the second end of the tubular structure.

13. The stent of claim 12, wherein the tertiary strut arrangement comprises one row of third struts, the third struts being interconnected within the row in a sinusoidal arrangement about the circumference of the tubular structure, wherein crests and troughs in the sinusoidal arrangement comprise connection points of the third struts.

14. The stent of claim 13, wherein the longitudinal struts disposed at an end of the primary strut arrangement and extending in a direction of the second end of the tubular structure are joined to the row of third struts at the crests, the crests in the row of third struts being closer to the second end than the troughs in the row, wherein eight circumferentially adjacent third struts are disposed between every two longitudinal struts joined to the row, the tertiary strut arrangement thereby being connected with the primary strut arrangement, and further wherein the sinusoidal arrangement of the row of third struts is 180 degrees out of phase with the sinusoidal arrangements of the rows of first struts of the primary strut arrangement.

15. The stent of claim 12, wherein the tertiary strut arrangement comprises:
two rows of third struts, the third struts being interconnected within each row in a sinusoidal arrangement about the circumference, wherein crests and troughs in the sinusoidal arrangement comprise connection points of the third struts, wherein the sinusoidal arrangements of the two rows of third struts are in phase with each other, and wherein a second row of the two rows is closer to the second end of the tubular structure than a first row of the two rows; and
axial struts connecting the two rows of third struts from crests of the first row to crests of the second row, the crests in each row being closer to the second end than the troughs in the row, wherein, in each of the two rows, eight circumferentially adjacent third struts are disposed between every two axial struts joined to the row, the axial struts being substantially parallel to a longitudinal axis of the tubular structure.

16. The stent of claim 15, wherein the longitudinal struts disposed at an end of the primary strut arrangement and extending in a direction of the second end of the tubular structure are joined to the first row of third struts at troughs spaced apart from every axial strut by three third struts or five third struts, thereby connecting the primary strut arrangement with the tertiary strut arrangement, and wherein the sinusoidal arrangements of the two rows of third struts are in phase with the sinusoidal arrangements of the rows of first struts of the primary strut arrangement.

17. The stent of claim 1, wherein any two circumferentially adjacent first struts have a wishbone shape when the stent is expanded, wherein the primary strut arrangement extends over at least 80% of the length of the structure from the first end to the second end, the length of the stent being at least about 50 mm, and wherein the longitudinal struts are substantially parallel to a longitudinal axis of the tubular structure, the tubular structure including two or more radiopaque markers secured to the first end and to the second end thereof.

18. The stent of claim 17, further comprising a secondary strut arrangement disposed adjacent to the primary strut arrangement at the first end of the tubular structure, wherein the secondary strut arrangement comprises a row of second struts, the second struts being interconnected within the row in a sinusoidal arrangement about the circumference, wherein crests and troughs in the sinusoidal arrangement comprise connection points of the second struts, wherein the sinusoidal arrangement of the row of second struts is in phase with the sinusoidal arrangements of the rows of struts of the primary strut arrangement, and wherein the longitudinal struts at an end of the primary strut arrangement and extending in a direction of the first end of the tubular structure are joined to the row of second struts at the troughs, the troughs in the row of second struts being closer to the first end than the crests in the row, eight circumferentially adjacent second struts being disposed between every two longitudinal struts joined to the row of second struts, thereby connecting the primary strut arrangement with the secondary strut arrangement.

19. The stent of claim 18, further comprising a tertiary strut arrangement including at least one row of third struts disposed adjacent to the primary strut arrangement at the second end of the tubular structure.

20. The stent of claim 19, wherein the tertiary strut arrangement comprises one row of third struts, the third struts being interconnected within each row in a sinusoidal arrangement about the circumference of the tubular structure, wherein crests and troughs in the sinusoidal arrangement comprise connection points of the third struts,
wherein the longitudinal struts disposed at an end of the primary strut arrangement and extending in a direction of the second end of the tubular structure are joined to the row of third struts at the crests, the crests in the row of third struts being closer to the second end than the troughs in the row, wherein eight circumferentially adjacent third struts are disposed between every two longitudinal struts joined to the row, the tertiary strut arrangement thereby being connected with the primary strut arrangement, and further wherein the sinusoidal arrangement of the row of third struts is 180 degrees out of phase with the sinusoidal arrangements of the rows of first struts of the primary strut arrangement.
